(19) United States
(12) Patent Application Publication
(10) Pub. No.: US 2007/0098640 A1
(43) Pub. Date: May 3, 2007

(54) Nanoparticle-based imaging agents for X-ray/computed tomography

(75) Inventors: Peter John Bonitatebus JR., Saratoga Springs, NY (US); Oskar Hans Erik Axelsson, Loma (SE); Amit Mohan Kulkarni, Clifton Park, NY (US); Brian Christopher Bales, Niskayuna, NY (US); Deborah Joy Walter, Burnt Hills, NY (US); Andrew Soliz Torres, Clifton Park, NY (US); Chiaki Treynor, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 11/265,686

(22) Filed: Nov. 2, 2005

(51) Int. Cl. A61K 49/04

(52) U.S. Cl. 424/9.42; 977/928

(57) ABSTRACT

The present invention is generally directed to core/shell nanoparticles, wherein such core/shell nanoparticles comprise a nanoparticle core and a nanoshell disposed about the nanoparticle core such that, in the aggregate, they form a core/shell nanoparticle that is operable for use as an imaging agent in X-ray/computed tomography (CT). Typically, such core/shell nanoparticle-based X-ray CT imaging agents further comprise a targeting species for targeting the imaging agent to diseased sites.

### TECHNICAL FIELD

The present invention relates generally to imaging agents for use in X-ray/computed tomography, and more specifically to nanoparticle-based imaging agents.

### BACKGROUND INFORMATION

Iodinated benzoic acid derivatives continue to serve as standard X-ray/computed tomography (CT) imaging agents, despite the risk factors and side effects associated with intravenous iodine injection. Additionally, such standard CT imaging agents are typically of low molecular weight, and they are known to clear from the human body very rapidly, making it difficult to target these agents to disease sites (Shi-Bao Yu and Alan D. Watson, Chem. Rev. 1999, 99, 2353-2377).

The literature describes experimental nanoparticle systems containing gadolinium (Gd) for CT imaging. However, in such systems, only a relatively small number of gadolinium atoms may be delivered to/in the vicinity of the target tissues. Such approaches include a liposomal approach, in which iodinated molecules are encapsulated into liposomes (Leike et al., Invest. Radiol. 2001, 36(6), 303-308), as well as a dendritic approach, in which gadolinium atoms are conjugated to G-4 Starburst® polyamidoamide (PAMAM) dendrimers (Yordanov et al., Nano Letters 2002, 2(6), 595-599). Both approaches deliver, at most, a couple hundred heavy metal (i.e., gadolinium) atoms.

Efforts to deliver a greater number of heavy metal atoms have included the use of nanoparticles of such heavy metals. See PCT International Publication Nos. WO 03/075961 A2 and WO 2005/051435 A2. Although nanoparticles of elemental (zerovalent) metal species have the highest density (number of heavy metal atoms/volume), they suffer from issues such as robust synthesis and stability from oxidation. Nanoparticles of inert metals such as gold (e.g., such as described in WO 03/075961 A2) can overcome these issues, but are not very cost effective.

As a result of the foregoing, there is a continuing need for new imaging agents for CT, especially to the extent that such imaging agents can provide for improved performance and benefit in one or more of the following areas: robust synthesis, reduced cost, image contrast enhancement, increased blood half-life, decreased toxicity, decreased radiation dose, and targeting capability.

### BRIEF DESCRIPTION OF THE INVENTION

The present invention is generally directed to core/shell nanoparticles, wherein such core/shell nanoparticles comprise a nanoparticle core and a nanoshell disposed about the nanoparticle core such that, in the aggregate, they form a core/shell nanoparticle that is operable for use as an imaging agent in X-ray/computed tomography (CT).

In some embodiments, the present invention is directed to an imaging agent of a first type comprising a passive nanoparticle core and an active nanoshell, the nanoshell comprising an active CT contrast agent material and being disposed about the nanoparticle core such that in the aggregate they form a core/shell nanoparticle that is operable for use as an imaging agent in CT imaging.

In some embodiments, the present invention is directed to an imaging agent of a second type comprising an active nanoparticle core and an active nanoshell, the nanoshell being materially different from that of the nanoparticle core and comprising an active CT contrast agent material, and being disposed about the nanoparticle core such that in the aggregate they form a core/shell nanoparticle that is operable for use as an imaging agent in CT imaging.

In some embodiments, the present invention is directed to an imaging agent of a third type comprising an active nanoparticle core and a passive nanoshell, the nanoshell being disposed about the nanoparticle core such that in the aggregate they form a core/shell nanoparticle that is operable for use as an imaging agent in CT imaging.

In some embodiments, the present invention is directed to methods of making any of the above-described imaging agents. In some or other embodiments, the present invention is directed to methods of using such imaging agents in CT.

The present invention uses a nanoparticle approach to deliver a relatively large number of high-density, highly-attenuating (radio-opaque molecular structures with effective atomic number greater than or equal to Z=34, the atomic number of selenium) atoms in elemental or molecular form to improve CT contrast enhancement. In some embodiments, the present invention provides for targeting of specific disease sites by the CT imaging agent. In some embodiments, the present invention provides for macrophage uptake of the CT imaging agent. In some embodiments, the present invention provides for a CT imaging agent with increased blood half-life.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 generally depicts a cross-sectional view of a core/shell nanoparticle so as to illustrate the relationship between the components;

FIG. 2 depicts a cross-sectional view of a core/shell nanoparticle of the first type comprising a passive core and an active shell, in accordance with some embodiments of the present invention;

FIG. 3 depicts a cross-sectional view of a core/shell nanoparticle of the second type comprising an active core and an active shell, in accordance with some embodiments of the present invention;

FIG. 4 depicts a cross-sectional view of a core/shell nanoparticle of the third type comprising an active core and a passive shell, in accordance with some embodiments of the present invention;

FIG. 5 illustrates, in flow diagram form, a method of using core/shell nanoparticles as imaging agents for computed tomography, in accordance with some embodiments of the present invention;

FIG. 6 is a transmission electron microscopy (TEM) image of a passive core/active shell nanoparticle with an iron oxide core and iodinated shell, in accordance with some embodiments of the present invention;

FIG. 7 is a TEM image of an active core largely comprising hafnium oxide, in accordance with some embodiments of the present invention;

FIG. 8 is a TEM image of active core/passive shell nanoparticles comprising a tungsten oxide core and polymeric shell, in accordance with some embodiments of the present invention; and

FIG. 9 is a computed tomography (CT) image of active core/passive shell nanoparticles with a tungsten oxide core and polymeric shell, in accordance with some embodiments of the present invention.

### DETAILED DESCRIPTION OF THE INVENTION

The present invention is generally directed to core/shell nanoparticles, wherein such core/shell nanoparticles comprise a nanoparticle core and a nanoshell disposed about the nanoparticle core such that, in the aggregate, they form a core/shell nanoparticle that is operable for use as an X-ray imaging agent, particularly in computed tomography (CT).

Referring to the drawings in general, it will be understood that the illustrations are for the purpose of describing a particular embodiment of the invention and are not intended to limit the invention thereto.

While most of the terms used herein will be recognizable to those of skill in the art, the following definitions are nevertheless put forth to aid in the understanding of the present invention. It should be understood, however, that when not explicitly defined, terms should be interpreted as adopting a meaning presently accepted by those of skill in the art.

“Computed Tomography,” abbreviated “CT,” as defined herein and also known as computed axial tomography or computer-assisted tomography (CAT) and body section roentgenography, is a medical imaging method employing tomography where digital processing is used to generate a three-dimensional image of the internals of an object (or subject) from a large series of two-dimensional X-ray images taken around a single axis of rotation. While the discussion herein focuses on computed tomography, it will be appreciated by those of skill in the art that such discussions apply generally to all types of X-ray imaging.

“Imaging agents,” as defined herein and also known as contrast agents, are agents that comprise a material that can significantly attenuate incident X-ray radiation causing a reduction of the radiation transmitted through the volume of interest. After undergoing CT image reconstruction and typical post-processing, this increased X-ray attenuation is interpreted as an increase in the density of the volume of interest, which creates a contrast enhancement in the volume comprising the contrast agent relative to the background tissue in the image. Because the discussion herein is generally applicable to all forms of X-ray imaging, the imaging agents of the present invention are generally referred to herein as “X-ray/computed tomography imaging agents.” This term is used interchangeably with “computed tomography (CT) agents.”

In reference to the core and shell components of a core/shell nanoparticle, the terms “active” and “passive” refer to the components' ability to create a contrast enhancement in CT imaging. A conventional CT scanner scans uses a broad spectrum of X-ray energy between about 10 keV and about 140 keV. Those skilled in the art will recognize that the amount of X-ray attenuation passing through of a particular material per unit length is expressed as the linear attenuation coefficient. At an X-ray energy spectrum typical in CT imaging, the attenuation of materials is dominated by the photoelectric absorption effect and the Compton Scattering effect. Furthermore, the linear attenuation coefficient is well known to be a function of the energy of the incident X-ray, the density of the material (related to molar concentration), and the atomic number (Z) of the material. For molecular compounds or mixtures of different atoms the ‘effective atomic number,’ Z_{eff}, can be calculated as a function of the atomic number of the constituent elements. The effective atomic number of a compound of known chemical formula is determined from the relationship:

$${Z}_{eff} = {[ {\sum }_{k = 1}^{P} {w}_{{f}_{k}} {Z}_{k}^{β} ]}^{1 / β} \qquad ( Eq . 1 )$$

where Zₖ is the atomic number of simple elements, P is the total quantity of simple elements, and w_{f}_{ₖ} is the weight fraction of simple elements with respect to the total molecular weight of the molecule (related to the molar concentration). The optimal choice of the incident X-ray energy for CT imaging is a function of the size of the object to be imaged and is not expected to vary much from the nominal values. It is also well known that the linear attenuation coefficient of the contrast agent material is linearly dependent on the density of the material, i.e., the linear attenuation coefficient can be increased if the material density is increased or if the molar concentration of the contrast material is increased. However, the practical aspects of injecting contrast agent material into patients, and the associated toxicity effects, limit the molar concentration that can be achieved. Therefore it is reasonable to separate potential contrast agent materials according to their effective atomic number. Based on simulations of the CT contrast enhancement of typical materials for a typical CT energy spectrum with a molar concentration of approximately 50 mM, Applicants estimate that materials with effective atomic number greater than or equal to 34 will yield appropriate contrast enhancement of about 30 Hounsfield units (HU), or 3% higher contrast than water. Therefore, Applicants have defined potential CT contrast materials as being either passive, having an effective atomic number of less than 34 (i.e., Z_{eff}<34), or active, having an effective atomic number greater than or equal to 34 (i.e., Z_{eff}≧34). See, e.g., Chapter 1 in Handbook of Medical Imaging, Volume 1. Physics and Psychophysics, Eds. J. Beutel, H. L. Kundel, R. L. Van Metter, SPIE Press, 2000).

A “nanoparticle,” as defined herein, is a particle with an average diameter of between about 1 nm and about 500 nm, and can be used to refer to nanoparticle cores and core/shell nanoparticle aggregates. Such nanoparticles can be spherical or irregularly shaped, and, particularly on the smaller end of this size range, are differentiated from molecular organometallic complexes. A “nanoshell,” as defined herein, is the shell region of the core/shell nanoparticle. Such a nanoshell is disposed about the nanoparticle core in such a way that it may or may not conform to the topography of the underlying nanoparticle core.

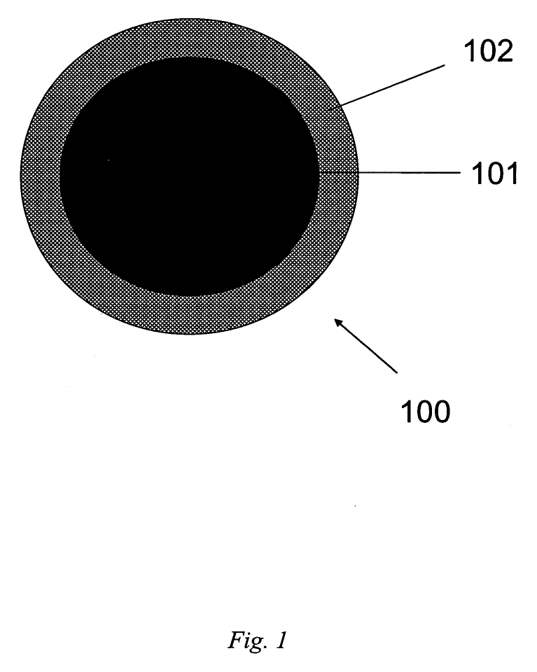

As mentioned above, and referring to FIG. 1, in some embodiments the present invention is directed toward an imaging agent comprising: (a) a nanoparticle core 101; and (b) a nanoshell 102, the nanoshell being disposed about the nanoparticle core such that, in the aggregate, they form a core/shell nanoparticle 100 that is operable for use as an imaging agent in computed tomography (generally, X-ray) imaging. While FIG. 1 depicts a cross-section of a perfectly spherical core/shell nanoparticle, such core/shell nanoparticles can also be irregularly-shaped.

In some embodiments, the above-described imaging agent further comprises at least one targeting agent. Such targeting agents are useful for targeting the imaging agents to specific diseased regions of a subject's body. Typically, the targeting agent is an antibody (e.g., IgG) or other peptide, but can also be a nucleic acid (e.g., DNA, RNA) or other suitable chemical species. Generally, the at least one targeting agent is attached to one or both of the nanoparticle core and the nanoshell disposed about the nanoparticle core. Such attachment typically comprises a linkage such as, but not limited to, a peptide linkage, a disulfide linkage, an isothiourea linkage, an isourea linkage, a sulfonamide linkage, an amine linkage, a carbamate linkage, an amidine linkage, a phosphoramidate linkage, a thioether linkage, an arylamine linkage, an aryl thioether linkage, an ether linkage, a hydrazone linkage, traizole linkage, oxime linkage, and combinations thereof. See, e.g., Chapter 2 in Bioconjugate Techniques. G. T. Hermanson, Academic Press, 1996.

In some embodiments, the above-described CT imaging agents are optimized for macrophage uptake via control of the core/shell nanoparticle surface charge and/or the presentation of functional groups which induce macrophage uptake (e.g., polyvinyl sulfate).

For the core/shell nanoparticles described herein for use as imaging/contrast agents in CT, the nanoparticle core typically has an average diameter of from about 1 nm to about 100 nm, more typically from about 2 nm to about 100 nm, and most typically from about 2 nm to about 20 nm. The nanoshell disposed about the nanoparticle core typically has an average thickness of from about 0.5 nm to about 100 nm. Accordingly, the aggregate of the nanoparticle core and the nanoshell of the imaging agent typically has an average diameter of from about 2 nm to about 500 nm, more typically from about 2 nm to about 100 nm, and most typically from about 5 nm to about 20 nm.

1. Core/Shell Nanoparticles of a First Type

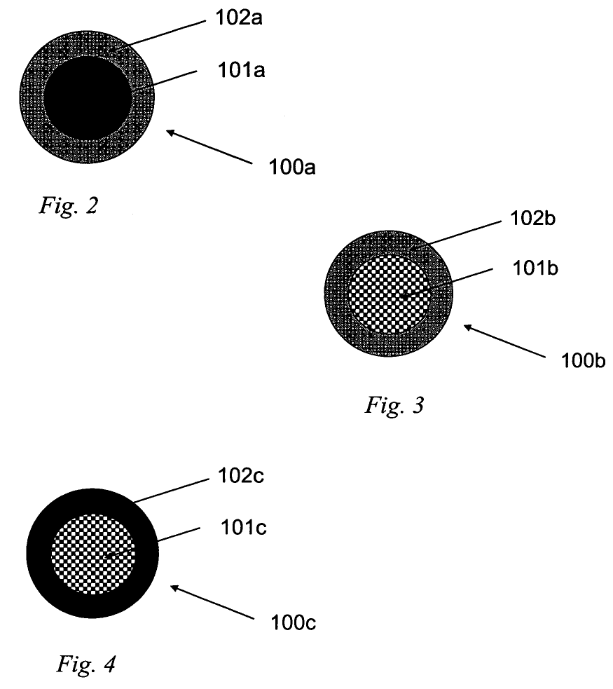

Referring to FIG. 2, in some embodiments, the present invention is directed to an imaging agent of a first type 100a comprising a passive nanoparticle core 101a and an active nanoshell 102a, the nanoshell comprising an active CT contrast agent material and being disposed about the nanoparticle core such that in the aggregate (i.e., collectively) they form a core/shell nanoparticle 100a that is operable for use as an imaging agent in CT imaging.

The material of which the above-described nanoparticle core 101a is comprised is not particularly limited, as this nanoparticle core does not generally comprise active contrast agent material (Z_{eff}<34). Generally, however, the nanoparticle core comprises a material distinguishable from that of the nanoshell. In some embodiments, the nanoparticle core comprises material such as, but not limited to, non-heavy metals and their alloys, oxides, halides, nitrides, sulfides, borides, phosphides, carbides, and the like; polymers; ceramics; and combinations thereof. Suitable such materials include, but are not limited to, iron (Fe), aluminum (Al), silicon (Si), magnesium (Mg), and their respective oxides.

The nanoshell 102a can comprise any material that suitably provides for an active nanoshell for use as a contrast agent in CT, and which can be suitably disposed about the nanoparticle core. Generally, such materials comprise active materials (Z_{eff}≧34) that can suitably interact with X-rays in a manner so as to enhance contrast in CT imaging. Generally, such active materials comprise elements of high atomic weight (e.g., heavy metals). Suitable such active materials include, but are not limited to, iodine (I), gadolinium (Gd), tungsten (W), tantalum (Ta), hafnium (Hf), bismuth (Bi), and combinations thereof.

In some of the above-described embodiments, the active material is disposed directly about the core. In some or other embodiments, the active material is associated with a passive component of the nanoshell. In a particular embodiment, such association can involve active nanoparticles being dispersed within a polymer matrix disposed about the nanoparticle core, wherein the active nanoparticles and the polymer matrix collectively make up the nanoshell. In some other embodiments, the nanoshell comprises a molecular coating that complexes or otherwise associates with active material. Suitable such molecular coatings include, but are not limited to, coatings of ligands, polymers, clusters, carbohydrate species, and combinations thereof.

2. Core/Shell Nanoparticles of a Second Type

Referring to FIG. 3, in some embodiments, the present invention is directed to an imaging agent of a second type 100b comprising an active nanoparticle core 101b and an active nanoshell 102b, the nanoshell being materially different from that of the nanoparticle core, comprising an active CT contrast agent material, and being disposed about the nanoparticle core such that in the aggregate they form a core/shell nanoparticle 100b that is operable for use as an imaging agent in CT imaging.

The material of which the above-described nanoparticle core 101b is comprised is not particularly limited, except that this nanoparticle core generally comprises active contrast agent material (i.e., Z_{eff}≧34). Generally, the nanoparticle core comprises a material distinguishable from that of the nanoshell. The nanoparticle core typically comprises material such as, but not limited to, metals and their alloys, oxides, halides, nitrides, carbides, sulfides, phosphides, selenides, tellurides, borides, and combinations thereof.

The nanoshell 102b can comprise any material that suitably provides for an active nanoshell (i.e., Z_{eff}≧34) for use as a contrast agent in CT, and which can be suitably disposed about the nanoparticle core. Generally, such materials comprise active materials that can suitably interact with X-rays in a manner so as to enhance contrast in CT imaging. Generally, such active materials comprise elements of high atomic weight (e.g., heavy metals). Suitable such materials include, but are not limited to, iodine (I), gadolinium (Gd), tungsten (W), tantalum (Ta), bismuth (Bi), hafnium (Hf), and combinations thereof.

As in the case of CT imaging agents of the first type, in some of the above-described embodiments, the active material is disposed directly about the core. In some or other embodiments, the active material is associated with a passive component of the nanoshell. In a particular embodiment, such association can involve active nanoparticles being dispersed within a polymer matrix disposed about the nanoparticle core, wherein the active nanoparticles and the polymer matrix collectively make up the nanoshell. In some other embodiments, the nanoshell comprises a molecular coating that complexes or otherwise associates with active material. Suitable such molecular coatings include, but are not limited to, coatings of ligands, polymers, clusters, carbohydrate species, and combinations thereof.

3. Core/Shell Nanoparticles of a Third Type

Referring to FIG. 4, in some embodiments, the present invention is directed to an imaging agent of a third type 100c comprising an active nanoparticle core 101c and a passive nanoshell 102c, the nanoshell being disposed about the nanoparticle core such that in the aggregate they form a core/shell nanoparticle 100c that is operable for use as an imaging agent in CT imaging.

The material of which the above-described nanoparticle core 101c is comprised is generally limited only in that it not be metallic and that it at least comprise an active material for contrast enhancement in CT (Z_{eff}≧34). Generally, the nanoparticle core comprises a material distinguishable from that of the nanoshell. The nanoparticle core typically comprises material such as, but not limited to, metal oxides, metal nitrides, metal carbides, metal sulfides, metal phosphides, metal borides, metal selenides, metal tellurides, and combinations thereof.

The material of which the nanoshell 102c is comprised is not particularly limited, but generally does not comprise active CT contrast agent material (Z_{eff}<34) and must generally be capable of being disposed about the nanoparticle core. Suitable such materials include, but are not limited to, ligands, polymer, cluster species, carbohydrate species, and combinations thereof.

4. Methods of Using

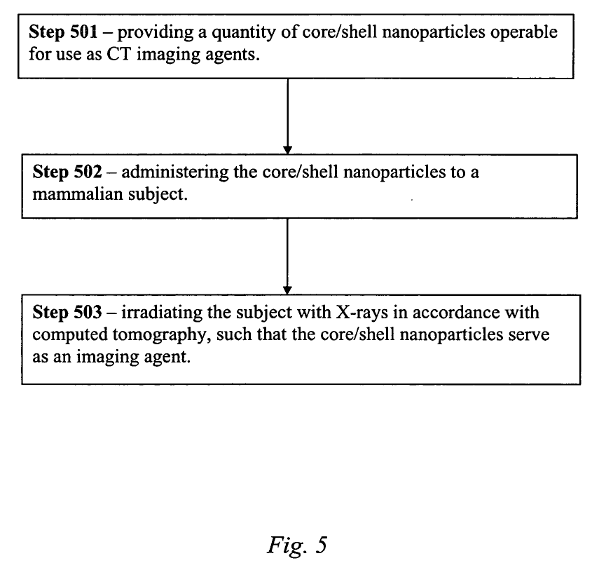

In some embodiments, the present invention is directed to methods of using any or all of the above-described types of imaging agents in CT applications. Referring to FIG. 5, such methods typically comprise the steps of: (Step 501) providing a quantity of core/shell nanoparticles comprising an active computed tomography contrast agent material, the core/shell nanoparticles each comprising a nanoparticle core and a nanoshell disposed about the nanoparticle core; (Step 502) administering the core/shell nanoparticles to a mammalian subject; and (Step 503) irradiating the subject with X-rays, in accordance with computed tomography, such that the core/shell nanoparticles serve as an imaging agent. In many such embodiments, such core/shell nanoparticles further comprise targeting agents such that, as imaging agents, they can be targeted to specific diseased areas of the subject's body. In some embodiments, the above-described core/shell nanoparticles, to the extent that they persist in the blood, are blood pool agents.

In some such above-described methods of using the above-described imaging agents, the core/shell nanoparticles provide for a CT signal of generally at least about 5 Hounsfield units to at most about 5000 Hounsfield units, and more particularly at least about 100 Hounsfield units to at most about 5000 Hounsfield units.

The following examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLE 1

This Example serves to illustrate how a first type of CT imaging agent can be prepared, in accordance with some embodiments of the present invention. In this particular Example, the CT imaging agent comprises an active shell of triiodobenzene-based ligand linked to a passive core such as poly(styrene) or iron oxide nanoparticles.

Synthesis of the Passive Core

A 25 mL, 3-neck Schlenk flask was fitted with a condenser, stacked on top of a 130 mm Vigreux column, and a thermocouple was introduced. The condenser was fitted with a nitrogen inlet and nitrogen flowed through the system. The Schlenk flask and Vigreux column were insulated with glass wool. Trimethylamine N-oxide (TMAO) (0.570 g, 7.6 mmol) and oleic acid (OA) (0.565 g, 2.0 mmol) were dispersed in 10 mL of dioctylether (OE). The dispersion was heated to 80° C. at a rate near 20° C./min. Once the mixture had reached 80° C., 265 μL of Fe(CO)₅ (2.0 mmol) was rapidly injected into the stirring solution via the Schlenk joint. The solution turned black instantaneously, with a violent production of a white “cloud.” The solution rapidly heated to ˜120-140° C. The reaction pot cooled to 100° C., a temperature at which it was kept and stirred for 75 minutes.

After stirring at 100° C. for 75 minutes, the temperature was increased to 280° C. at a rate near 20° C./min. After the solution stirred at 280° C. for 75 minutes, the heating mantel and glass wool were removed to allow the reaction to return to room temperature. Once at room temperature, an aliquot was removed and dissolved in toluene for sizing by dynamic (laser) light scattering (DLS) and transmission electron microscopy (TEM) imaging analysis (TEM). Half of the remaining solution (˜5 mL) was added to 20 mL of acetonitrile. The acetonitrile/OE mixture was stirred well and then centrifuged at 3000 rpm for 5 minutes to separate the layers. Oftentimes, the acetonitrile layer was observed to have a faint yellow color. The acetonitrile layer was discarded and the OE layer was used for ligand exchange chemistry. To prepare a sample for inductively coupled plasma-atomic emission spectroscopy (ICP-AES) measurement, the other half of the crude reaction solution was added to 20 mL of isopropanol, and the solution was centrifuged for 10 minutes at 3000 rpm. The supernatant was decanted, an additional 20 mL of isopropanol was added, and again the precipitate was collected by centrifugation. The precipitated iron oxide nanoparticles were allowed to air-dry overnight.

Synthesis of the Active Shell

In some embodiments there is a coupling to —OH functionality on the surface of the core nanoparticles, such as iron oxide. In one such exemplary case, 3-amino-2,4,6-triiodobenzoic acid (Aldrich) was reacted with mono-mesolated PEG-750 monomethyl ether in the presence of base to add one or two PEG-750 monomethyl ether moieties onto the amino functionality so as to impart water solubility to the molecule. This was subsequently treated with oxallyl chloride or thionyl chloride in the presence of base to create the acid chloride, then treated with 3-aminopropyltriethoxysilane. The product, after purification, was reacted with the surface of, for example, an iron oxide nanoparticle to covalently bond the iodine-containing ligand through siloxane linkages.

Alternatively, in some embodiments, the above-described shell material is coupled to carboxylic acid or amine surface-modified polystyrene nanoparticles. Carboxylic acid surface-modified polystyrene nanoparticles (e.g., from Bangs Beads, Fishers, Ind.) are reduced to the corresponding alcohol using LiAlH₄ and then reacted with the acid chloride in the example synthesis above. This yields polystyrene particles with the same iodinated shell as above. Alternatively, amino-modified polystyrene beads can be used to directly conjugate to the acid chloride without further modification.

In another exemplary embodiment, where there is a coupling to —OH functionality on the surface of particles, Omnipaque® (Iohexol) (GE Healthcare) is treated with 1 equivalent of p-toluene sulfonyl chloride then with base to epoxidize one or more of the vicinal diol functionalities in iohexol. This is subsequently treated with 3-aminopropyltriethoxysilnae (Gelest) to provide functionality for covalently bonding to, for example, an iron oxide nanoparticle though siloxane linkages.

In another embodiment involving a coupling to carboxylic acid or amine surface modified polystyrene nanoparticles, carboxylic acid surface modified polystyrene nanoparticles can be reduced, as described above, to the corresponding alcohol. Treatment with base followed by iohexol epoxide (see above) affords iohexol-coated polystyrene nanoparticles. Similarly, amine surface-modified nanoparticles can be reacted with iohexol epoxide.

A solution containing iron oxide nanoparticles coated with oleic acid (0.75 mg Fe), 3-amino-2,4,6-triiodobenzoic acid (0.305 g, 0.6 mmol), and sodium methoxide (32 mg, 0.6 mmol) dissolved in tetrahydrofuran (THF) (5 mL) was sonicated for 20 h to yield a solution of iron oxide nanoparticles coated with 3-amino-2,4,6-triiodobenzoic acid.

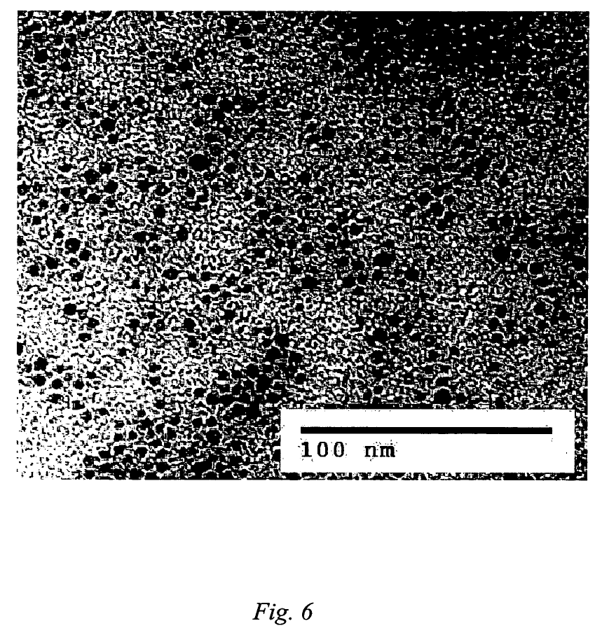

FIG. 6 is a transmission electron microscopy (TEM) image of passive core/active shell nanoparticles with an iron oxide core and iodinated shell, in accordance with some embodiments of the present invention.

### EXAMPLE 2

This Example serves to illustrate how a second type of CT imaging agent can be prepared, in accordance with some embodiments of the present invention. In this particular Example, an active shell comprising triiodobenzene-based covalent ligands is linked to an active core of hafnium oxide.

Synthesis of the Active Core

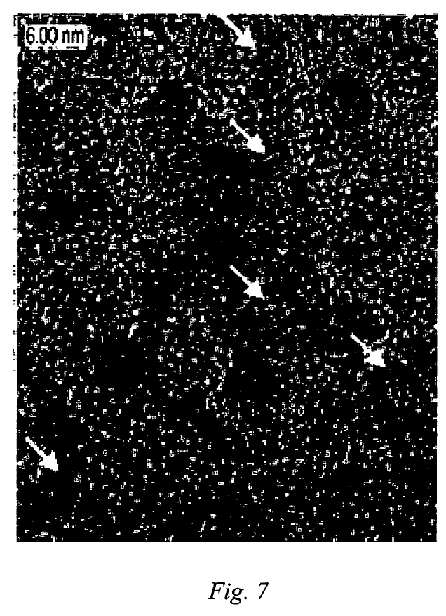

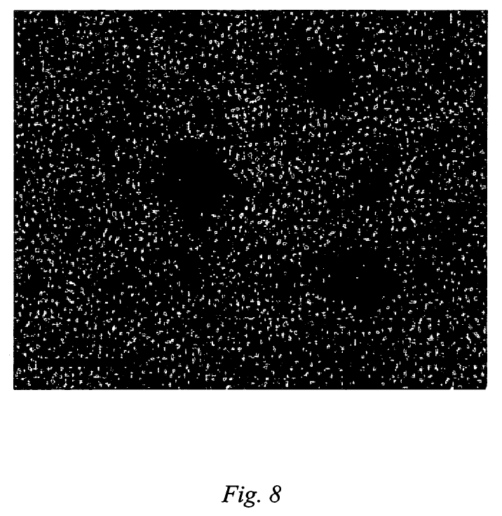

Hafnia nanocrystals (HfO₂) may be prepared from a suspension of hafnium oxychloride in ethanol. An organosilane-based coating may be applied as follows: dilute 3-glycidoxypropyl(trimethoxysilane), GPTS, using butanol (volume ratio 1:0.5) and perform a pre-hydrolysis step by addition of 0.1M HCl keeping the molar ratio of GPTS:H₂O at 1:0.5. Subject the resulting solution to vigorous stirring overnight at room temperature, then load with the HfO₂ nanocrystals. See Ribeiro et al., Appl. Phys. Lett. 2000, 77 (22), 3502-3504. FIG. 7 is a TEM image of an active core largely comprising hafnium oxide, in accordance with some embodiments of the present invention.

Synthesis of the Active Shell

In some embodiments, this involves coupling to OH functionality on the surface of the hafnium oxide cores. In one such instance, 3-Amino-2,4,6-triiodobenzoic acid (Aldrich) is reacted with mono-mesolated PEG-750 in the presence of base to add one or two PEG-750 moieties onto the amino functionality to impart water solubility to the molecule. This is subsequently treated with oxallyl chloride or thionyl chloride in the presence of base to create the acid chloride, then treated with 3-aminopropyltriethoxysilane (Gelest). The product, after purification, is reacted with the surface of, for example, an hafnium oxide core to covalently bond the iodine-containing ligand through siloxane linkages.

In some or other embodiments, Omnipaque® (Iohexol) (GE Healthcare) is treated with 1 equivalent of p-toluene sulfonyl chloride, then with base to epoxidize one or more of the vicinal diol functionality in iohexol. This is subsequently treated with 3-aminopropyltriethoxysilnae (Gelest) to provide functionality for covalently bonding to, for example, an hafnium oxide core though siloxane linkages.

### EXAMPLE 3

This Example serves to illustrate how a third type of CT imaging agent can be prepared, in accordance with some embodiments of the present invention. In this particular Example, a passive shell linked to an active core of hafnium oxide.

Hafnia nanocrystals (HfO₂) may be prepared from a suspension of hafnium oxychloride in ethanol (see EXAMPLE 2). An organosilane-based coating may be applied as follows: dilute 3-glycidoxypropyl(trimethoxysilane), GPTS, using butanol (volume ratio 1:0.5) and perform a pre-hydrolysis step by addition of 0.1M HCl keeping the molar ratio of GPTS:H₂O at 1:0.5. The resulting solution is subjected to vigorous stirring overnight at room temperature, then loaded with the HfO₂ nanocrystals. See Ribeiro et al., Appl. Phys. Lett. 2000, 77 (22), 3502-3504. FIG. 8 is a TEM image of active core/passive shell nanoparticles comprising a tungsten oxide core and polymeric shell, in accordance with some embodiments of the present invention.

### EXAMPLE 4

This Example serves to illustrate how a core/shell nanoparticle-based CT imaging agent can be used to enhance contrast, in accordance with some embodiments of the present invention.

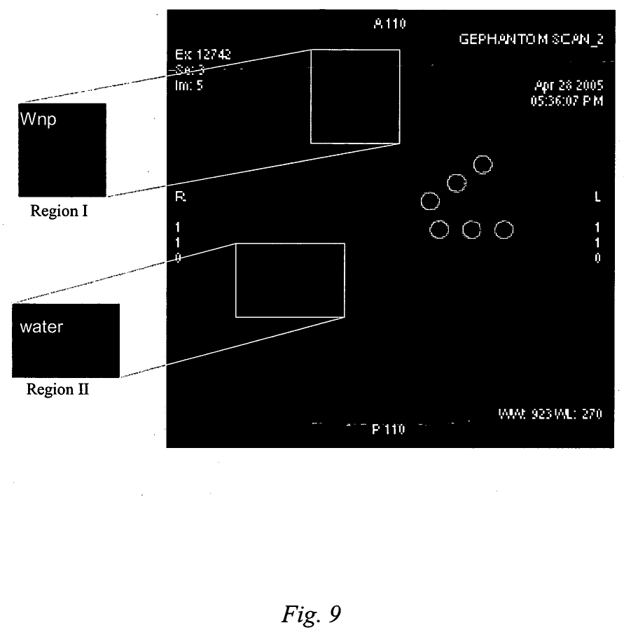

In order to enhance a volume of interest in a CT image, Applicants used active core/passive shell nanoparticles (i.e., CT imaging agents of the third type) with a tungsten oxide core and polymeric shell mixed in water. A test phantom was constructed from a cylindrical block of Lucite®, approximately 15 cm in diameter, with cylindrical cavities with diameters approximately 1.5 cm arranged circularly. These cavities were filled with a mixture of water and one other potential contrast agent material at various concentration levels. A CT image was acquired using a standard medical CT scanner (GE Lightspeed), with an energy spectrum of 120 kVp, and a source current of 195 mAs. The image was reconstructed with a slice thickness of 1.25 mm using a standard reconstruction kernel. The example image from the experiment is displayed in FIG. 9. Since water closely represents the CT enhancement of most soft tissue in the human body, the window and level of the display was set in order to visually inspect the relative contrast of the tungsten nanoparticle contrast agent mixed with water, with respect to pure water. In the image displayed in FIG. 9, the window width was set to ˜1000 HU and the window level was ˜270 HU. In FIG. 9, Region II of the image is shown where the cavity is filled with pure water. In FIG. 9, Region I of the image is shown where the cavity is filled with tungsten oxide core and polymeric shell nanoparticles mixed with water at a molar concentration of ˜94 mM. The enhancement due to the contrast agent is seen as a brighter disk. In order to quantify the level of contrast enhancement, Applicants used a standard medical workstation (GE Advantage Workstation) to compute the mean and standard deviation of the CT image HU values in the regions of the pure water and the tungsten nanoparticle contrast agent mixed with water. Applicants found that the mean CT number of the water was approximately 0 HU, as expected, and the mean CT number of the tungsten nanoparticle contrast agent mixed with water at approximately 70 mM was approximately 400 HU. This represents an increase in contrast which is acceptable for most CT imaging applications.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. An X-ray/computed tomography imaging agent comprising:
a) a passive nanoparticle core; and
b) an active nanoshell, comprising an active computed tomography contrast agent material, disposed about the nanoparticle core such that, in the aggregate, they form a core/shell nanoparticle that is operable for use as an imaging agent in X-ray/computed tomography imaging.

2. The X-ray/computed tomography imaging agent of claim 1, further comprising at least one targeting agent.

3. The X-ray/computed tomography imaging agent of claim 2, wherein the at least one targeting agent is attached to a core/shell nanoparticle component selected from the group consisting of the nanoparticle core, the shell disposed about the nanoparticle core, and combinations thereof.

4. The X-ray/computed tomography imaging agent of claim 1, wherein the nanoparticle core has an average diameter of from about 1 nm to about 100 nm, and wherein the nanoshell disposed about the nanoparticle core has an average thickness of from about 0.5 nm to about 100 nm.

5. The X-ray/computed tomography imaging agent of claim 1, wherein the aggregate of the nanoparticle core and the nanoshell has an average diameter of from about 2 nm to about 500 nm.

6. The X-ray/computed tomography imaging agent of claim 1, wherein the nanoparticle core comprises material selected from the group consisting of elements with Z_{eff}<34.

7. The X-ray/computed tomography imaging agent of claim 1, wherein the nanoparticle core comprises material selected from the group consisting of non-heavy metals, and their alloys, oxides, halides, nitrides, sulfides, borides, selenides, phosphides, and carbides; polymers; ceramics; and combinations thereof.

8. The X-ray/computed tomography imaging agent of claim 1, wherein the nanoshell comprises active material selected from the group consisting of elements with Z_{eff}≧34.

9. The X-ray/computed tomography imaging agent of claim 1, wherein the nanoshell comprises active material selected from the group consisting of iodine, gadolinium, tungsten, tantalum, hafnium, bismuth, and combinations thereof.

10. The X-ray/computed tomography imaging agent of claim 8, wherein the nanoshell further comprises a passive material component within which the active material is dispersed.

11. The X-ray/computed tomography imaging agent of claim 8, wherein the nanoshell further comprises a passive material component with which the active material is associated, and wherein the passive material component is selected from the group consisting of ligands, polymers, clusters, carbohydrate species, and combinations thereof.

12. The X-ray/computed tomography imaging agent of claim 2, wherein the targeting agent is selected from the group consisting of a peptide, a nucleic acid, and combinations thereof.

13. The X-ray/computed tomography imaging agent of claim 2, wherein the targeting agent is an antibody.

14. The X-ray/computed tomography imaging agent of claim 2, wherein the targeting agent is IgG.

15. The X-ray/computed tomography imaging agent of claim 3, wherein the targeting agent is attached via a linkage selected from the group consisting of a peptide linkage, a disulfide linkage, an isothiourea linkage, an isourea linkage, a sulfonamide linkage, an amine linkage, a carbamate linkage, an amidine linkage, a phosphoramidate linkage, a thioether linkage, an arylamine linkage, an aryl thioether linkage, an ether linkage, a hydrazone linkage, triazole linkage, oxime linkage, and combinations thereof.

16. A method comprising the steps of:
a) providing a quantity of core/shell nanoparticles comprising:
i) a passive nanoparticle core; and
ii) an active nanoshell, comprising an active computed tomography contrast agent material, disposed about the nanoparticle core such that they collectively form a core/shell nanoparticle;
b) administering the core/shell nanoparticles to a mammalian subject; and
c) irradiating the mammalian subject with X-rays, in accordance with computed tomography, such that the core/shell nanoparticles serve as an imaging agent.

17. The method of claim 16, wherein use of core/shell nanoparticles as an imaging agent for computed tomography relies, in part, on an ability of cells present in specific tissue to recognize the core/shell nanoparticles.

18. The method of claim 16, wherein the core/shell nanoparticles further comprise a targeting agent, and wherein use of core/shell nanoparticles as an imaging agent for computed tomography relies, in part, on an ability of the targeting agent to target specific cells.

19. The method of claim 16, wherein the core/shell nanoparticles persist in the blood such that they are blood pool agents.

20. The method of claim 18, wherein the targeting agent is selected from the group consisting of a peptide, a nucleic acid, and combinations thereof.

21. The method of claim 18, wherein the targeting agent is an antibody.

22. The method of claim 18, wherein the targeting agent is IgG.

23. The method of claim 18, wherein the targeting agent is attached to a core/shell nanoparticle component selected from the group consisting of the nanoparticle core, the nanoshell disposed about the nanoparticle core, and combinations thereof.

24. The method of claim 16, wherein the core/shell nanoparticle provides for a CT signal of at least about 100 Hounsfield units to at most about 5000 Hounsfield units.
